SEQUENCE LISTING (24 sequences)

SEQ ID NO: 1 - protein, 183 aa, Homo sapiens
RLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQ

SEQ ID NO: 2 - protein, 661 aa, Homo sapiens
GPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 3 - protein, 92 aa, Homo sapiens
VPPLVKVTHHVTSSVTTLRCRALNYYPQNITMKWLKDKQPMDAKEFEPKDVLPNGDGTYQGWITLAVPPGEEQRYTCQVEHPGLDQPLIVIW

SEQ ID NO: 4 - protein, 753 aa, Artificial sequence
  Lactoferin / HFE-based recycling domain RC1
GPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKVPPLVKVTHHVTSSVTTLRCRALNYYPQNITMKWLKDKQPMDAKEFEPKDVLPNGDGTYQGWITLAVPPGEEQRYTCQVEHPGLDQPLIVIW

SEQ ID NO: 5 - protein, 275 aa, Artificial sequence
  Lactoferin / HFE-based recycling domain RC2
RLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTCQVEHPGLDQPLIVIW

SEQ ID NO: 6 - protein, 753 aa, Artificial sequence
  Lactoferin / HFE-based recycling domain RC3
VPPLVKVTHHVTSSVTTLRCRALNYYPQNITMKWLKDKQPMDAKEFEPKDVLPNGDGTYQGWITLAVPPGEEQRYTCQVEHPGLDQPLIVIWGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 7 - protein, 275 aa, Artificial sequence
  Lactoferin / HFE-based recycling domain RC4
VPPLVKVTHHVTSSVTTLRCRALNYYPQNITMKWLKDKQPMDAKEFEPKDVLPNGDGTYQGWITLAVPPGEEQRYTCQVEHPGLDQPLIVIWRLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQ

SEQ ID NO: 8 - protein, 21 aa, Mus musculus
METDTLLLWVLLLWVPGSTGD

SEQ ID NO: 9 - protein, 20 aa, Homo sapiens
METPAQLLFLLLLWLPDTTG

SEQ ID NO: 10 - protein, 301 aa, Homo sapiens
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVH

SEQ ID NO: 11 - protein, 1042 aa, Artificial sequence [sequence omitted]

SEQ ID NO: 12 - protein, 597 aa, Artificial sequence [sequence omitted]

SEQ ID NO: 13 - protein, 1042 aa, Artificial sequence [sequence omitted]

SEQ ID NO: 14 - protein, 597 aa, Artificial sequence [sequence omitted]

SEQ ID NO: 15 - protein, 251 aa, Homo sapiens
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSS

SEQ ID NO: 16 - protein, 992 aa, Artificial sequence
  CFP-RC1(n)TNF
METDTLLLWVLLLWVPGSTGDGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKVPPLVKVTHHVTSSVTTLRCRALNYYPQNITMKWLKDKQPMDAKEFEPKDVLPNGDGTYDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSS

SEQ ID NO: 17 - protein, 547 aa, Artificial sequence
  CFP-RC2(n)TNF
METDTLLLWVLLLWVPGSTGDRLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTCQVEHPGLDQPLIVIWDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSS

SEQ ID NO: 18 - protein, 992 aa, Artificial sequence [sequence omitted]

SEQ ID NO: 19 - protein, 547 aa, Artificial sequence
  CFP-RC2(c)TNF
METDTLLLWVLLLWVPGSTGDDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSRLLRSHSLHYLFMGASEQDLGLSLFEALGYVDDQLFVFYDHESRRVEPRTPWVSSRISSQMWLQLSQSLKGWDHMFTVDFWTIMENHNHSKESHTLQVILGCEMQEDNSTEGYWKYGYDGQDHLEFCPDTLDWRAAEPRAWPTKLEWERHKIRARQNRAYLERDCPAQLQQLLELGRGVLDQQVPPLVKVTHHVTSSVTTLRCRALNYYPQNITMKWLKDKQPMDAKEFEPKDVLPNGDGTYQGWITLAVPPGEEQRYTCQVEHPGLDQPLIVIW

SEQ ID NO: 20 - protein, 169 aa, Homo sapiens
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGGKGGLCQSSL

SEQ ID NO: 21 - protein, 910 aa, Artificial sequence
  CFP-RC1(n)IL18
METDTLLLWVLLLWVPGSTGDGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKVPPLVKVTHHVTSSVTTLRCRALNYYPQNITMKWLKDKQPMDAKEFEPKDVLPNGDGTYTPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGGKGGLCQSSL

SEQ ID NO: 22 - protein, 465 aa, Artificial sequence [sequence omitted]

SEQ ID NO: 23 - protein, 910 aa, Artificial sequence [sequence omitted]

SEQ ID NO: 24 - protein, 465 aa, Artificial sequence [sequence omitted]

(19) United States
(12) Patent Application Publication
(10) Pub. No.: US 2007/0264236 A1
(43) Pub. Date: Nov. 15, 2007

(54) Novel Therapeutic Fusion Proteins

(75) Inventor: Meija Yang, Scituate, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (NL)

(21) Appl. No.: 10/566,929

(22) PCT Filed: Aug. 4, 2004
(86) PCT No.: PCT/EP04/51715

§ 371 Date: Aug. 31, 2006

Related U.S. Application Data
(60) Provisional application No. 60/492,397, filed on Aug. 4, 2003.

(51) Int. Cl. A61K 38/17; A61K 48/00; C07H 21/04; C07K 14/705; C12N 15/63; C12N 5/10

(52) U.S. Cl. 424/93.2; 435/254.2; 435/320.1; 435/325; 435/348; 514/2; 530/402; 536/23.1

(57) ABSTRACT

The present invention provides novel therapeutic molecules called Culling Fusion Proteins (CFPs) that allow the continuous removal of therapeutic targets from extracellular space by exploiting the endosome/lysosome intracellular degradation pathway, and the exocytotic pathway in a combined manner. The products of the invention, by appropriately utilizing the cellular endocytosis and exocytosis mechanism, can be recycled multiple times by cells to eliminate undesired molecules, therefore such therapeutic molecules can be administered at low concentration.

### FIELD OF THE INVENTION

The present invention is directed to novel therapeutic proteins, compositions, and use of such proteins.

### BACKGROUND OF THE INVENTION

Recombinant therapeutic proteins function generally as agonists or antagonists to therapeutic targets, either circulating or located on the cellular membranes, that trigger responses into biological systems. In particular, the elimination of extracellular therapeutic targets (ETTs, from now on) can be achieved by binding to recombinant therapeutics such as soluble or decoy receptors, antibodies, or other binding proteins, that consequently block the disease pathways in which the ETT plays a crucial role. An example is provided by immunoadhesins, fusion proteins containing an ETT binding portion of protein linked to the Fc portion of human immunoglobulin s (WO 91/08298, WO 98/02540).

Such antagonists are often administered at high concentration in order to achieve the expected clinical outcomes by removing the circulating therapeutic target of endogeneous or exogenous origin. Side effects consequent to the high dosage often leads to the failure of the candidate drug molecules in the clinical development. Therefore, molecules that can degrade ETTs and possess multiple turnover numbers for neutralization processes are of high therapeutic interest.

A first category of neutralizing molecules is represented by enzymes, e.g. proteases, capable of modifying and/or degrading therapeutic targets in the extracellular space, inactivating them. Several classes of extracellular proteases have been characterized, such as MMPs (Matrix metalloproteinases; McCawley L J and Matrisian L M, 2001 ) or ADAMs (A Disintegrin And Metalloprotease; Blobel C P, 2002), in terms of substrate specificity but their activities cannot precisely and easily directed to a specific ETT.

A possible alternative is to redirect ETTs from extracellular fluids, such as blood or lymph, into intracellular compartments forming the endolysosomal system, wherein ETTs can be degraded by intracellular proteases. The endolysosomal system comprises a series of membrane-bound intracellular compartments, within which extracellular material flow vectorially, proceeding through a series of vescicle-like organelles, the main ones being the early endosome, the endosome carrier vesicle, the late endosome and the lysosome. The different components of the endolysosomal system are competent for specific proteolytic activities, and the whole process is highly dependent from the calcium concentration and the pH inside the vescicles (Pillay C S et al., 2002; Sachse M et al., 2002).

Extracellular material can enter the endolysosomal system by endocytosis or phagocytosis. Endocytosis constitutes an essential process in the regulation of the expression of cell surface molecules and receptors and receptor-mediated endocytosis is the sole cellular mechanism allowing the entrance of specific extracellular molecules, for modulating signaling pathways, introducing some metabolites, and/or degrade the bound molecule. The complexes formed by extracellular ligands and surface exposed receptors can enter the endolysosomal system and can be sorted within the early or late endosomes into one of three pathways:
(i) the entire ligand—receptor complex may be recycled back to the plasma membrane;
(ii) the ligand—receptor complex may dissociate, with the receptor being recycled to the cell surface and the ligand directed further along the pathway; or
(iii) the entire ligand-receptor complex may be targeted to the later stages of the pathway.

Receptor-mediated transport mechanisms provide a pathway for the trafficking of extracellular macromolecules into (endocytosis), outside (exocytosis), and across (transcytosis) the cell.

Amongst the various receptor-mediated transport mechanisms identified in recent years for the intracellular targeting and delivery of drugs (Swaan P W, 1998), the Transferrin receptor-mediated endocytosis pathway is one of the most studied (Qian Z M et al., 2002), and many molecules have been generated for this scope, such as transferrin-radioactive isotope conjugates, transferrin-toxin conjugates, as well as transferrin-DNA conjugates.

Transferrin receptor (TfR) is a dimeric membrane receptor that binds to serum transferrins. At pH 7.4, as on the cell surface, ferric Transferrin (Tf-Fe; chelated to iron) binds to TfR, and the complex is internalised via receptor-mediated endocytosis (Richardson D R and Ponka P, 1997). Tf-Fe-TfR complexes concentrate in an area called coated pits and, through the formation of clathrin-coated vesicles, they are internalised, forming endosomes. An ATP-driven proton-pump acidifies the interior of the endosomes, and the ferric ions are released from the Tf, likely through conformational changes of the Tf. Apo-transferrin (without iron) is tightly bound to TfR at pH 5.6, and is re-directed to the plasma membranes via budding of the early endosomes and exocytosis pathway. Thus the Apo-transferrin (Apo Tf) and ferric transferrin (Tf-Fe) possess different binding characteristics to TfR. Once Tf/TfR complex reached cell surface, the TfR undergoes conformational changes and releases the Apo-transferrin from the binding. The cycle is completed with the release of Transferrin into the circulation.

Transferrin receptors can be recognized by other proteins that are members of the transferrin family of proteins are involved in Fe³⁺ transport (serum transferrins), in particular lactoferrin and Hereditary Hemochromatosis protein.

Lactoferrin (Lf) is a broadly expressed iron-binding protein involved in host defense against infection and severe inflammation. Lactoferrin also binds to cell surface receptors and transport irons into the cells, but, unlike Tf-TfR complex, lactoferrin is not exocytosed. However, both apo- and ferric lactoferrin, which allows delivery of iron to the small intestine, can specifically bind and be endocytosed (McAbee D D et al., 1993). Lactoferrin is very similar to transferrin in the three-dimensional structure and well as sites for iron binding. Lactoferrin distinguishes from transferrin in its iron-releasing activity (at a pH comprised between 2 and 4, and not from 6 to 4 as for Transferrin), and additional activities, such as proteolytic, cell growth promoting, and ant microbial activities (Baker E N et al., 2002). The receptor-mediated cellular transport of lactoferrin has been demonstrated in different models, such as cultured differentiated bovine brain capillary endothelial cells (Fillebeen C et al., 1999), or rat liver (Meilinger M et al., 1995).

Hereditary hemochromatosis protein (HFE) was identified as the product of a gene defective in the hereditary iron-overload. HFE has been characterized as regulator for the iron-uptake, although the mechanism of the regulation is not clear. The HFE protein binds to TfR tightly at pH 7.4, but not at pH 6.0, and it is transported with the transferrin receptor in endocytic compartments (Lebron J A et al., 1998; Davies P S et al., 2003). The soluble domains of this protein had been co-crystalized with TfR. The resolution of the structure revealed that alpha1-alpha2 domain of HFE binds to the TfR (Bennett M J et al., 2000). Although the mechanism of its regulatory function on TfR remains unknown, it is suggested that the HFE is released from TfR in endosomes due to the low pH. The alpha3 domain of the HFE protein interacts with beta2-macroglobulin via a disulfide bond, and this interaction is required for exocytosis of the HFE protein to the cell surface (Feder J N et al., 1998).

Many structure-function studies have been done on proteins belonging to the Transferrin family. For example, chimeric proteins consisting of segments derived from human lactoferrin and bovine transferrin have been generated in order to delineate the binding region on the human lactoferrin for various bacterial receptors (Wong H and Schryvers A B, 1998). Alternatively, Transferrin fusion proteins have been designed to deliver therapeutic molecules, such as nerve growth factor (NGF), to the central nervous systems through the blood-brain barrier (Park E et al., 1998).

Lactoferrin variants having altered, pH-dependent iron binding and release but unaltered receptor binding properties are known (WO 97/45136). Other lactoferrin mutants exhibit reduced glycosylation and an increased serum half-life, also due to the reduced iron and receptor binding, and can be fused to therapeutic proteins or peptides (WO 03/20746). The selective transport of therapeutic, bi-specific chimeric proteins containing Transferrin (WO 91/12023, WO 96/39510), peptides (WO 02/44329) or alpha1-alpha3 domain of HFE (WO 02/24929) into cells have been disclosed, but no active means to promote the exocytosis thus the re-use of the chimeric molecules are disclosed herein.

### SUMMARY OF THE INVENTION

The present invention provides novel therapeutic molecules called Culling Fusion Proteins (CFPs) based on specific domains of HFE protein that allow the continuous removal of therapeutic targets from extracellular space by exploiting the endosome/lysosome intracellular degradation pathway, and the exocytotic pathway in a combined manner. The products-of the invention, by appropriately utilizing the cellular endocytosis and exocytosis mechanism, can be recycled multiple times by cells to eliminate undesired molecules, therefore such therapeutic molecules can be administered at low concentration.

Other objects of the present invention relates to the DNA encoding the HFE-based chimeric proteins, cells expressing them, and method for producing, isolating, assaying, and using such proteins. Further features and advantages of the invention, such as pharmaceutical compositions and methods for and treatment of diseases, will be apparent from the following detailed description.

### DESCRIPTION OF THE FIGURES

FIG. 1: representation of the mechanism by which Culling Fusion Proteins (CFPs) allow the removal of a the target molecules (ETT) from extracellular space and to degrade them through lysosomes. The CFP and cell membrane receptors are then transported to the cell surface and become available for the next round of the culling cycle.

FIG. 2: (A) schematic structure of CFPs, composed of protein domain binding to an extracellular therapeutic target and called culling domain (CD), and a recycing domain which comprises an Exocytosis Domain (ExDO) and an Endocytosis Domain (EnDO). (B) schematic structure of the CFPs exemplifying the invention, which are based on recycling domains containing human deltaN-lactoferrin (dN-Lf), alpha3 domain of human HFE (HFE-a3), or alpha1-alpha2 domain of human HFE (HFE-a1a2). The Culling Domain for VEGF is formed by the Ig-like domains 1-3 of VEGFR-1 (VEGFR-1 d1-3). The Culling Domain for TNF is formed by the soluble portion of TNF receptor I called TNF binding protein I (TNFbp-1). The Culling Domain for IL-18 is formed by the IL-18 binding protein I (IL18bp).

The black box indicates the heterologous signal sequence of mouse Ig kappa chain V-III.

FIG. 3: example of experimental design for a cell-based assay validating CFPs, by demonstrating the transcytosis of CFPs in cells that are seeded on a porous support included in a bicameral chamber.

### DETAILED DESCRIPTION OF THE INVENTION

The main object of the present invention is a chimeric protein comprising:
a) a recycling domain capable of binding the human cell surface receptor and formed by an Exocytosis Domain and an Endocytosis Domain; and
b) a protein domain binding an Extracellular Therapeutic Target.

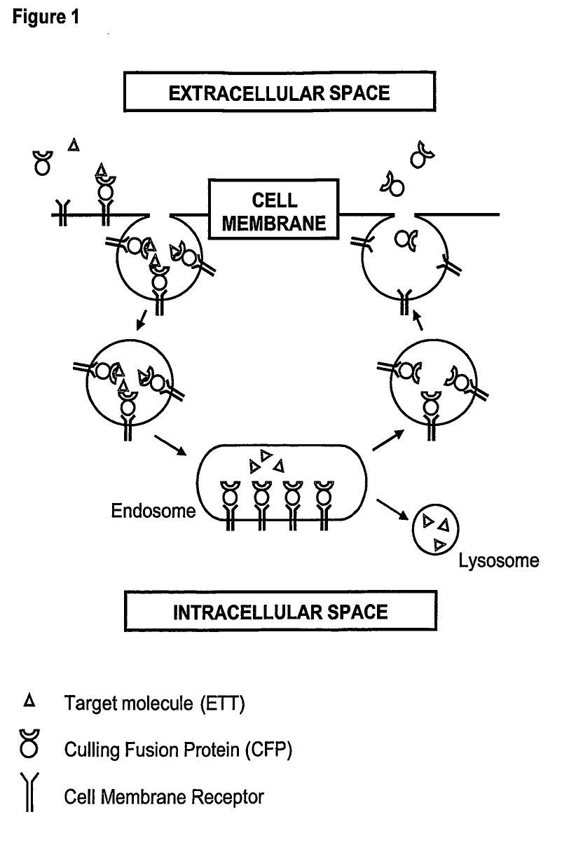

Chimeric proteins of the present invention, called Culling Fusion Proteins (CFPs), include at least three components which can be assembled in different order: a Culling Domain (CD), an Exocytosis Domain (ExDO) and an Endocytosis Domain (EnDO). The Culling Domain comprises a polypeptide sequence binding the ETT. The Exocytosis Domain comprises a polypeptide sequence binding a cell surface receptor expressed on one or more types of somatic cells. The Endocytosis Domain comprises a polypeptide sequence capable of routing the CFP to the cell surface after the dissociation from the cell receptor and the ETT in the extracellular space (FIG. 1).

Endosome-lysosome formation upon receptor-mediated endocytosis is a natural pathway that degrades much of the blood stream molecules, including EGF, insulin, cholera toxin, virus particles, and LDL. The present invention takes advantage of this degradation pathway to neutralize therapeutic targets. Such catalytic degradation may minimize the dose of drug molecules as they can be used repetitively, and may reduce build-up of neutralizing antibodies and/or side effects.

In view of the literature mentioned above, the human Transferrin receptor is a human cell receptor that can be used for recycling the chimeric proteins of the invention. Therefore, preferred Endocytosis and Exocytosis domain forming the recycling domain should interact with human Transferrin system.

In this context, examples of Endocytosis domain can be chosen amongst sequences such as the alpha1-alpha2 domain of human HFE (fragment 23-205 of SWISSPROT Acc. No. Q30201; SEQ ID NO: 1) and human deltaN-Lactoferrin (fragment 51-711 of SWISSPROT Acc. No. P02788; SEQ ID NO: 2). These Endocytosis domains interacts with the human Transferrin receptor and can be fused to an Exocytosis domain formed by the alpha3 domain of human HFE protein (fragment 206-297 of SWISSPROT Acc. No. Q30201; SEQ ID NO: 3). This latter sequence allows the CFP to bind to membrane protein such as beta2-Microglobulin at the acidic pH of the endosome and to be brought to the cell surface for the exocytosis.

The human Lactoferrin and HFE variants disclosed in the literature show therapeutic features limited to improved serum half -life, in vitro solution stability, or bioavailability of the fusion molecules. The present invention describes the generation of fusion molecules acting in a very different way, i.e. that can function as a shuttle molecule to transport extracellular therapeutic targets into the cellular compartments for degradation and recycled in the extracellular space.

The Exocytosis and Endocytosis domain above mentioned can be assembled in the recycling domain in any order. The Lactoferin/HFE-based recycling domain RC1 (SEQ ID NO: 4) and RC2 (SEQ ID NO: 5) have the Endocytosis domain N-terminal to the Exocytosis domain. The Lactoferin/HFE-based recycling domain RC3 (SEQ ID NO: 6) and RC4 (SEQ ID NO: 7) have the Exocytosis domain N-terminal to the Endocytosis domain.

The Culling Domain (CD) is the CFP protein domain capable of binding a n Extracellular Therapeutic Target (ETT) with an affinity sufficient to allow the internalization of the CFP-ETT complex from the extracellular space to the intracellular endosomal system, via the Transferrin receptor in the specific case, so that the ETT can be released in the cell where it will maintained and, possibly, degraded in the hepatocytes or in any other cell type presenting the cell receptor recognized by the CFPs.

The ETT can be any endogenously- or exogeously-produced, natural or synthetic molecule circulating in the extracellular fluid, such as blood or lymph, found associated to a disease: a cytokine, a chemokine, a hormone, a growth factor, an immunoglobulin, a glycolipid, a glycosaminoglycan, a nucleic acid, a viral protein, a bacterial protein, or a synthetic organic molecule.

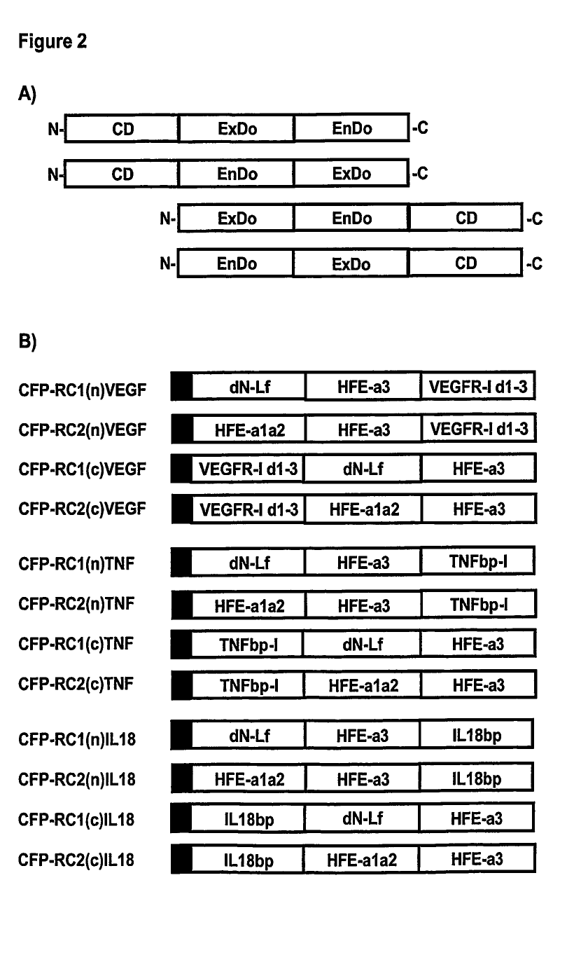

The CD can be fused at N- or C-terminus of the recycling domain (FIG. 2A) and can be a protein sequence selected from: an extracellular region of a membrane-bound protein, a secreted protein, a viral protein, an antigen binding domain of an antibody, or one or more selected domain of such protein sequences.

Examples of ETTs and of human proteins naturally binding the ETT and therefore containing a corresponding CD are shown in Table I. Alternatively, CD protein sequences can be identified into variable regions of monovalent antibodies, phage-displayed sequences, or any other library of protein sequence which are screened by the means of the ETTs, and which can be subcloned in a vector (Pini A and Bracci L, 2000). An alternative solution is provided by viral proteins known to interact with human cytokines and chemokines (Beisser P S et al., 2002).

The chimeric proteins of the present invention may further comprise an amino acid sequence belonging to a heterologous protein sequence other than the ones comprised in the proteins containing the Exocytosis Domain, the Endocytosis Domain, and the protein domain binding an Extracellular Therapeutic Target. This heterologous sequence is intended to provide additional properties without impairing significantly the antagonistic, “culling” activity.

Examples of such additional properties are an easier purification procedure (e.g. use of an histidine tag to allow affinity purification), a longer half-life in body fluids, or extracellular localization. This latter feature is of particular importance for defining a specific group of chimeric proteins included in the above definition since it allows CFPs to be localized in the space where not only where the isolation and purification of these peptides is facilitated, but also where CFPs, ETTs and cell receptor naturally interact. Therefore, if the order of CD and of the recycling domain does not allow any naturally present signal sequence to be located at the N-terminus, the CFPs may comprise an heterologous signal peptide, such as the one of the mouse Ig kappa chain V-III (fragment 1-21 of SWISSPROT Acc. NO. P01658; SEQ ID NO: 8) or of the corresponding human sequence (fragment 1-21 of SWISSPROT Acc. NO. P18136; SEQ ID NO: 9).

The term “heterologous”, when used herein, is intended to designate any polypeptide belonging to a protein other than any of the ones whose specific domains are comprised in the CFP.

Example of heterologous sequences, that can be comprised in the soluble fusion proteins either at N- or at C-terminus, are the following: extracellular domains of membrane-bound protein, immunoglobulin constant regions (Fc region), multimerization domains, domains of extracellular proteins, signal sequences, export sequences, or sequences allowing purification by affinity chromatography.

Many of these heterologous sequences are commercially available in expression plasmids since these sequences are commonly included in the fusion proteins in order to provide additional properties without 2003). Examples of such additional properties are a longer lasting half-life in body fluids, the extracellular localization, or an easier purification procedure as allowed by the a stretch of Histidines forming the so-called “histidine tag” (Gentz et al., 1989) or by the “HA” tag, an epitope derived from the influenza hemagglutinin protein (Wilson et al., 1994). If needed, the heterologous sequence can be eliinated by a proteolytic cleavage, for example by inserting a proteolytic cleavage site between the soluble protein and the heterologous sequence, and exposing the purified soluble fusion protein to the appropriate protease. These features are of particular importance for the soluble fusion proteins since they facilitate their production and use in the preparation of pharmaceutical compositions.

When the soluble fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of the substances of the invention and the immunoglobulin sequence. The resulting fusion protein has improved properties, such as an extended residence time in body fluids (half-life), increased specific activity, increased expression level, or the purification of the fusion protein is facilitated.

In a preferred embodiment, the soluble protein is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

On the basis of the above indicated protein elements, a series of exemplary CFPs have been designed (FIG. 2B).

A first group of CFPs is directed against VEGF (Vascular Endothelial Growth Factor), a molecule promoting the proliferation of endothelial cells, a mechanism triggering tumor development. The extracellular region of VEGF receptors are formed by seven immunoglobulin homology domains, of which the second and third are critical for ligand binding and the first three domains are necessary for establishment of full binding affinity (Jussila L and Alitalo K., 2002). A CD formed by the three N-terminal immunoglobulin homology domains of human VEGFR-1 (fragment 27-327 of SWISSPROT Acc. No. P17948; SEQ ID NO: 10) can be fused at the C-terminus of the recycling domain RC1 or RC2 forming CFP-RC1(n)VEGF (SEQ ID NO: 11) or CFP-RC2(n)VEGF (SEQ ID NO: 12). This CD can be alternatively positioned at the N-terminus of the recycling domain RC1 or RC2 forming CFP-RC1(c)VEGF (SEQ ID NO: 13) or CFP-RC2(c)VEGF (SEQ ID NO: 14).

A second group of CFPs is directed against TNFalpha (Tumor Necrosis Factor alpha), a molecule responsible of many autoimmune diseases. The soluble portion of TNF receptors, called Tumor necrosis factor binding protein, can be used for binding circulating TNFalpha and blocking the interaction with the membrane-bound receptors (Lorenz H M and Kalden J R, 2002). A CD formed by the Tumor necrosis factor binding protein 1 (fragment 41-291 of SWISSPROT Acc. No. P19438; SEQ ID NO: 15) can be fused at the C-terminus of the recycling domain RC1 or RC2 forming CFP-RC1(n)TNF (SEQ ID NO: 16) or CFP-RC2(n)TNF (SEQ ID NO: 17). This CD can be alternatively positioned at the N-terminus of the recycling domain RC1 or RC2 forming CFP-RC1(c)TNF (SEQ ID NO: 18) or CFP-RC2(c)TNF (SEQ ID NO: 19).

A third group of CFPs is directed against IL-18 (Interleukin 18), a potent proinflammatory cytokine that has pathophysiological roles in several inflammatory conditions. A protein called IL-18 binding protein (IL-18bp) can bind IL-18 and block its activities (Nakanishi K et al., 2001). A CD formed by IL-18bp (fragment 29-197 of SWISSPROT Acc. No. 095998; SEQ ID NO: 20) can be fused at the C-terminus of the recycling domain RC1 or RC2 forming CFP-RC1(n)IL18 (SEQ ID NO: 21) or CFP-RC2(n)IL18 (SEQ ID NO: 22). This CD can be alternatively positioned at the N-terminus of the recycling domain RC1 or RC2 forming CFP-RC1(c)IL18 (SEQ ID NO: 23) or CFP-RC2(c)IL18 (SEQ ID NO: 24).

The Exocytosis Domain, the Endocytosis Domain, and the protein domain binding an Extracellular Therapeutic Target forming a CFP can be also active mutants of the corresponding natural sequence. The properties of chimeric proteins of the present invention should be maintained, or even potentiated, in these resulting active mutants. This category of molecules includes natural or artificial analogs of said sequence, wherein one or more amino acid residues have been added, deleted, or substituted, provided they display the same biochemical activity as defined in the present invention at comparable or higher levels, and as determined by means known in the art and disclosed in the Examples below. For example, nested deletions can be generated in an element of a CFP in order to minimize the protein sequence needed for exert its activity and consequently reduce the dimension of the CFP.

In accordance with the present invention, preferred changes in these active mutants are commonly known as “conservative” or “safe” substitutions. Conservative amino acid substitutions are those with amino acids having sufficiently similar chemical properties, in order to preserve the structure and the biological function of the molecule. It is clear that insertions and deletions of amino acids may also be made in the above defined sequences without altering their function, particularly if the insertions or deletions only involve a few amino acids, e.g., under te n, and preferably under three, and do not remove or displace amino acids which are critical to the functional conformation of a protein or a peptide.

The literature provide many models on which the selection of conservative amino acids substitutions can be performed on the basis of statistical and physico-chemical studies on the sequence and/or the structure of natural protein (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The synonymous amino acid groups and more preferred synonymous groups are those defined in Table II.

Similar compounds may also result from conventional mutagenesis technique of the encoding DNA, from combinatorial technologies at the level of encoding DNA sequence (such as DNA shuffling, phage display/selection), from computer-aided design studies, or from incorporating unnatural amino acids, followed by the validation for the desired activities as described in the prior art and in the Examples below.

Alternatively, amino acids in the soluble proteins of the invention that are essential for function can also be identified by methods known in the art, such as site directed mutagenesis or alanine-scanning mutagenesis (Cunningham et al., 1989). Of special interest are substitutions of charged amino acids with other charged or neutral amino acids that may produce proteins with highly desirable improved characteristics, such as less aggregation. Aggregation may not only reduce activity but also be problematic when preparing pharmaceutical or physiologically acceptable formulations, because aggregates can be immunogenic (Cleland et al., 1993).

In the specific case of recycling domains interacting with Transferrin system, the natural iron binding sites present in HFE and Lactoferrin can be mutated in order to generate molecules that do not interfere with the cellular iron metabolism.

Alternatively, the active mutein may result from sequence alterations reducing the immunogenicity of said soluble protein when administered to a mammal. The literature provides many example on these sequence alterations that can be designed and introduced at this scope or for other functional optimizations that allow a safe and effective administration of a therapeutic protein, especially when it is non-human, non-mammalian, or non-natural protein (Vasserot A P et al., 2003; Marshall S A et al., 2003; Schellekens H, 2002; Gendel S M, 2002; Graddis T J et al., 2002; WO 03/104263; WO 03/006047; WO 02/98454; WO 02/96454; WO 0217941 5; WO 02/79232; WO 02/66514; WO 01/40281; WO 98/52976; WO 96/40792; WO 94/11028).

The chimeric protein of the present invention can be in alternative forms which can be preferred according to the desired method of use and/or production, for example in the form of an active fraction, precursor, salt, derivative, conjugate or complex.

The term “active” means that such alternative CFPs forms should maintain the functional features of the CFPs of the present invention containing natural sequences, and, according to any of the assay presented in the examples, has a comparable, or even increased, activity. Finally the CFPs should be as well pharmaceutically acceptable and useful.

By the activity being “comparable” is meant that the activity measured in any of the described assays for the variant of the soluble protein is at least of the same order of magnitude, and preferably 75%, 80%, 85%, 90%, 95%, 96%, 97%, 98%, 99%, or 100%, and not more than 101%, 102%, 103%, 104%, 105%, 110%, 115%, 120% or 125% of the activity measured using a corresponding CFP as defined by the present invention.

By the activity being “increased” is meant that the activity measured in any of the described assays for the variant of the soluble protein is at least 125%, 130%, 135%, 140%, 145%, 150%, 155%, 160%, 170%, 180%, 190%, 200%, 225%, 250%, 275%, 300%, 325%, 350%, 375%, 400%, 450%, or 500% of the activity measured using a corresponding CFP as defined by the present invention.

The term “fraction” refers to molecules resulting from modifications which do not normally alter primary sequence, for example in vivo or in vitro chemical derivativization of peptides (acetylation or carboxylation), those made by modifying the pattern of phosphorylation (introduction of phosphotyrosine, phosphoserine, or phosphothreonine residues), glycosylation (by exposing the peptide to enzymes which affect glycosylation e.g., mammalian glycosylating or deglycosylating enzymes), acetylation, amidation, and/or myristoylation.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the peptides, polypeptides, or analogs thereof, of the present invention. Salts of a carboxyl group may be formed by means known in the art and indude inorganic salts, for example, sodium, calcium, ammonium, ferric or zinc salts, and the like, and salts with organic bases as those formed, for example, with amines, such as tiethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the N-/ or C-terminal groups according to known methods. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups.

Useful conjugates or complexes of the chimeric proteins of the present invention can be generated using molecules and methods known in the art, for example, for protein detection (radioactive or fluorescent labels, biotin) or for drug delivery, such as polyethylene glycol and other natural or synthetic polymers (Pillai O and Panchagnula R, 2001).

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the polypeptide with consideration of effects on functional or antigenic domains of the polypeptide. There are a number of attachment methods available to those skilled in the art, e.g., in EP401384.

A CFP resistant to proteolysis can be generated by replacing a —CONH— peptide bond with one or more of the following: a (CH2NH) reduced bond; a (NHCO) retro inverso bond; a (CH2-O) methylene-oxy bond; a (CH2-S) thiomethylene bond; a (CH2CH2) carba bond; a (CO—CH2) cetomethylene bond; a (CHOH—CH2) hydroxyethylene bond); a (N—N) bound; a E-alcene bond; or a —CH═CH— bond. Thus, the invention also encompasses a soluble CD164 or a variant thereof in which at least one peptide bond has been modified as described above. In addition, amino acids have chirality within the body of either L or D. In some embodiments it is preferable to alter the chirality of the amino acids in order to extend half-life within the body. Thus, in some embodiments, one or more of the amino acids are preferably in the L configuration. In other embodiments, one or more of the amino acids are preferably in the D configuration.

The compounds of the invention may be prepared by any well known procedure in the art, including recombinant DNA-related technologies described above, and chemical synthesis technologies.

Another object of the invention are the DNA molecules comprising the DNA sequences for the chimeric proteins of the invention, including nucleotide sequences substantially the same.

“Nucleotide sequences substantially the same” includes all other nucleic acid sequences that, by virtue of the degeneracy of the genetic code, also code for the given amino acid sequences.

The invention also includes expression vectors th at comprise the DNA molecules above defined, wherein expression of said DNA is under the control of a promoter, as well as host cells transformed with such vectors and a process of preparation of the chimeric proteins of the invention, comprising culturing the transformed cells in an appropriate culture media, and collecting the expressed protein.

The DNA sequence coding for the different elements forming CFPs can be generated by PCR methods, modified using restriction enzymes, and ligated to be inserted into a suitable plasmid. The coding sequences can be chosen in order to have a codon usage that is optimal for the selected expression host, such as in E. coli (Kane J F, 1995).

Once formed, the expression vector is introduced into a suitable host cell, which then expresses the vector to yield the desired protein. Expression of any of the recombinant proteins of the invention as mentioned herein can be effected in eukaryotic cells (e.g. yeast, insect or mammalian cells) or prokaryotic cells, using the appropriate expression vectors. Any method known in the art can be employed.

For example the DNA molecules coding for the proteins obtained by any of the above methods are inserted into appropriately constructed expression vectors by techniques well known in the art. Double stranded cDNA is linked to plasmid vectors by homopolymeric tailing or by restriction linking involving the use of synthetic DNA linkers or blunt-ended ligation techniques: DNA ligases are used to ligate the DNA molecules, and undesirable joining is avoided by treatment with alkaline phosphatase.

In order to be capable of expressing the desired protein, an expression vector should also comprise specific nucleotide sequences containing transcriptional and translational regulatory information linked to the DNA coding the desired chimeric protein in such a way as to permit gene expression and production of the protein. In order to be transcribed, the gene should be preceded by a promoter recognized by RNA polymerase, to which the enzyme binds and thus initiates the transcription process. There are a variety of such promoters in use, which work with different efficiencies (strong and weak promoters).

For Eukaryotic hosts, different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated.

The DNA molecule comprising the nucleotide sequence coding for the protein of the invention is inserted into vector(s), having the operably linked transcriptional and translational regulatory signals, which is capable of integrating the desired gene sequences into the host cell.

The cells that have been stably transformed by the introduced DNA can be selected by also introducing one or more markers allowing for selection of host cells containing the expression vector. The marker may also provide for phototrophy to an auxotropic host, biocide resistance, e.g. antibiotics, or heavy metals such as copper, or the like. The selectable marker gene can either be directly linked to the DNA gene sequences to be expressed, or introduced into the same cell by co-transfection.

The expression vector is any of the mammalian, yeast, insect or bacterial expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence can be optimized for the particular expression organism into which the expression vector is introduced (U.S. Pat. No. 5,082,767; Gustafsson C et al., 2004).

Additional important factors for selecting a particular plasmid or viral vector include: the ease with which recipient cells that contain the vector, may be recognized and selected from those recipient cells which do not contain the vector; the number of copies of the vector which are desired in a particular host; and whether it is desirable to be able to “shuttle” the vector between host cells of different species. A recombinant vector according to the invention comprises, but is not limited to, a YAC (Yeast Artificial Chromosome), a BAC (Bacterial Artificial Chromosome), a phage, a phagemid, a cosmid, a plasmid, or even a linear DNA molecule which may consist of a chromosomal, non-chromosomal, semi-synthetic or synthetic DNA.

Generally, recombinant expression vectors will include origins of replication, selectable markers permitting transformation of the host cell, and a promoter derived from a highly expressed gene to direct transcription of a downstream structural sequence. The heterologous structural sequence is assembled in appropriate phase with translation initiation and termination sequences, and preferably a leader sequence capable of directing secretion of the translated protein into the periplasmic space or the extracellular medium. In a specific embodiment wherein the vector is adapted for transfecting and expressing desired sequences in mammalian host cells, preferred vectors will comprise an origin of replication in the desired host, a suitable promoter and enhancer, and also any necessary ribosome binding sites, polyadenylation sites, splice donor and acceptor sites, transcriptional termination sequences, and 5′-flanking non-transcribed sequences. DNA sequences derived from the SV40 viral genome, for example SV40 origin, early promoter, enhancer, splice and polyadenylation sites may be used to provide the required non-transcribed genetic elements.

Once the vector(s) or DNA sequence containing the construct(s) has been prepared, the vector(s) may be introduced into an appropriate host cell by any of a variety of suitable means: transformation, transfection, conjugation, protoplast fusion, electroporation, calcium phosphate-precipitation, direct microinjection, etc.

Host cells may be either prokaryotic or eukaryotic. Preferred are eukaryotic hosts, e.g. mammalian cells, such as human, monkey, porcine, mouse, rabbit, sheep, hamster, mouse or rat. The cells can be primary cells, or secondary, immortalized, cultured cell strains. Cells like Chinese Hamster Ovary (CHO) cells, because they provide post-translational modifications to protein molecules, including correct folding or glycosylation at correct sites. Furthermore, human cells expressing CFPs can be directly used. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast recognizes leader sequences on cloned mammalian gene products and secretes peptides bearing leader sequences (i.e., pre-peptides).

After the introduction of the vector(s), the host cells are grown in a selective medium, which selects for the growth of vector-containing cells. Expression of the cloned gene sequence(s) results in the production of the desired proteins.

These objects of the invention can be achieved by combining the disclosure provided by the present patent application on CFPs with the knowledge of common molecular biology techniques. Many reviews (Makrides S C, 1999) and books provides teachings on how to clone and produce recombinant proteins using vectors and Prokaryotic or Eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Examples of chemical synthesis technologies are solid phase synthesis and liquid phase synthesis. As a solid phase synthesis, for example, the amino acid corresponding to the C-terminus of the peptide to be synthetized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic add for the Boc method, and with TFA for the Fmoc method.

Purification of the recombinant or synthetic chimeric proteins of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and are produced and immobilized on a gel matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification. Finally, the identity of the recombinant or synthetic chimeric proteins can be verified by any appropriate technology, such as mass spectrometry.

Alternatively, the CFPs can be isolated from milk of transgenic animals expressing the CFPs applying any of the methods disclosed in the literature (Protein Purification Applications, A Practical Approach (New Edition), Edited by Simon Roe, AEA Technology Products and Systems, Biosciences, 50; U.S. Pat. No. 6,140,552).

The invention includes purified preparations of the chimeric proteins of the invention. Purified preparations, as used herein, refers to the preparations which are at least 1%, preferably at least 5%, by dry weight of the compounds of the invention.

A further object of the present invention is a pharmaceutical composition comprising the chimeric protein of the invention, or of the cells expressing a chimeric protein of the invention, as active ingredient. Another object of the present invention is the use of the chimeric proteins of the invention, or of the cells expressing a chimeric protein of the invention, as medicament, and in particular as active ingredient in pharmaceutical compositions (and formulated in combination with pharmaceutically acceptable carriers, excipients, stabilizers, or diluents) for treating or preventing a disease related to an undesirable activity of an ETT.

CFPs act as antagonists of the ETT to which they are directed. Given the large variety of ETTs that can be targeted by the chimeric proteins of the invention. Using the VEGF-directed CFPs exemplified above, the disease can be cancer, or an autoimmune or inflammatory disease, taking instead TNFalpha-directed CFPs.

The primary function of the immune system is to protect an individual against infection by foreign invaders such as microorganisms, it may happen that the immune system attacks the individual's own tissues, leading to pathologic states known as autoimmune diseases, which are frequently associated with inflammatory processes. An appropriate CFP may eliminate the ETT that triggers these processes.

A non-limitative list of disorders where a medicament or a pharmaceutical composition comprising a CFP, includes: multiple sclerosis, systemic lupus erythematosus, rheumatoid arthritis, juvenile idiopathic arthritis, psoriatc arthritis, osteoarthritis, spondylarthropathies, inflammatory bowel disease, endotoxemia, Crohn's disease, Still's disease, uveitis, Wegener's granulomatosis, Behcet's disease, scleroderma, Sjogren's syndrome, sarcoidosis, pyodema gangrenosum, polymyositis, dermatomyositis, myocarditis, psoriasis, systemic sclerosis, hepatitis C, allergies, allergic inflammation, allergic airway inflammation, chronic obstructive pulmonary disease (COPD), mesenteric infarction, stroke, ulcerative colitis, allergic asthma, bronchial asthma, mesenteric infarction, stroke, fibrosis, post-ischemic inflammation in muscle, kidney and heart, skin inflammation, glomerulonephritis, juvenile onset type I diabetes mellitus, hypersensitvity diseases, cancer, viral or acute liver diseases, alcoholic liver failures, tuberculosis, septic shock, HIV-infection, graft-versus-host disease (GVHD) and atherosclerosis.

Another object of the present invention is, therefore, the method for treating or preventing a disease comprising the administration of an effective amount of a chimeric protein of the invention or of the cells expressing a chimeric protein of the invention.

The pharmaceutical compositions may contain, in addition to the CFP, suitable pharmaceutically acceptable carriers, biologically compatible vehicles and additives which are suitable for administration to an animal (for example, physiological saline) and eventually comprising auxiliaries (like excipients, stabilizers or diluents) which facilitate the processing of the active compounds into preparations which can be used pharmaceutically. Such compositions can be eventually combined with another therapeutic composition acting synergically or in a coordinated manner with the chimeric proteins of the invention. Alternatively, the other composition can be based with a compound known to be therapeutically active against the specific disease (e.g. IFNbeta for multiple sclerosis). These compositions can further comprise an additional immunosuppressant or anti-inflammatory substance. Alternatively, the pharmaceutical compositions comprising the soluble can be combined into a “cocktail” for use in the various treatment regimens.

The pharmaceutical compositions may be formulated in any acceptable way to meet the needs of the mode of administration. For example, the use of biomaterials and other polymers for drug delivery, as well the different techniques and models to validate a specific mode of administration, are disclosed in literature (Luo B and Prestwich G D, 2001; Cleland J L et al., 2001).

An “effective amount” refers to an amount of the active ingredients that is sufficient to affect the course and the severity of the disease, leading to the reduction or remission of such pathology. The effective amount will depend on the route of administration and the condition of the patient.

“Pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution.

Any accepted mode of administration can be used and determined by those skilled in the art to establish the desired blood levels of the active ingredients. For example, administration may be by various parenteral routes such as subcutaneous, intravenous, epidural, topical, intradermal, intrathecal, direct intraventricular, intraperitoneal, transdermal (e.g. in slow release formulations), intramuscular, intraperitoneal, intranasal, intrapulmonary (inhaled), intraocular, oral, or buccal routes.

Other particularly preferred routes of administration are aerosol and depot formulation. Sustained release formulations, particularly depot, of the invented medicaments are expressly contemplated.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99 percent, preferably from about 20 to 75 percent of active compound together with the excipient. Compositions that can be administered rectally include suppositories.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain is otonicity (e.g. mannitol) or chemical stability (e.g. preservatives and buffers). The formulation is sterilized by commonly used techniques. For transmucosal administration, penetrants appropriate to the barrier to be permeated are used in the formulation. Such penetrants are generally known in the art.

Pharmaceutical or physiologically acceptable preparations that can be taken orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable gaseous propellant, e.g., carbon dioxide. In the case of a pressurized aerosol the dosage unit may be determined by providing a valve to deliver a metered amount. Capsules and cartridges of, e.g., gelatin, for use in an inhaler or insulator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in aqueous vehicles, and may contain formulatory agents such as suspending, stabilizing and/or dispersing agents. Alternatively, the active ingredient may be in powder or lyophilized form for constitution with a suitable vehicle, such as sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Additionally, the compounds may be delivered using a sustained release system, such as semipermeable matrices of solid hydrophobic polymers containing the therapeutic agent. Various sustained-release materials have been established and are well known by those skilled in the art. Sustained release capsules may, depending on their chemical nature, release the compounds for a few weeks up to over 100 days or one year.

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight. Ordinarily 1 to 40 milligrams per kilogram per day given in divided doses or in sustained release form is effectve to obtain the desired results. Second or subsequent administrations can be performed at a dosage, which is the same, less than, or greater than the initial or previous dose administered to the individual. According to the invention, the substances of the invention can be administered prophylactically or therapeutically to an individual prior to, simultaneously or sequentially with other therapeutic regimens or agents (e.g. multiple drug regimens), in a therapeutically effective amount. Active agents that are administered simultaneously with other therapeutic agents can be administered in the same or different compositions.

For any compound used in the method of the invention, the therapeutically effective dose can be estimated initially from cell culture assays. For example, a dose can be formulated in animal models to achieve a circulating concentration range that includes or encompasses a concentration point or range shown to decrease cytokine expression in an in vitro system. Such information can be used to more accurately determine useful doses in humans. A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD50, (the dose lethal to 50% of the test population) and the ED50 (the dose therapeutically effective in 50% of the population). The dose ratio between toxic and therapeutic effects is the therapeutic index and it can be expressed as the ratio between LD50 and ED50. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED50, with little or no toxicity. The dosage may vary within this range depending upon the dosage form employed and the route of administration utilized. The exact formulation, route of administration and dosage can be chosen by the individual physician in view of the patients condition.

The term “treating” as used herein refers to administering a compound after the onset of clinical symptoms.

The term “preventing” as used herein refers to administering a compound before the onset of clinical symptoms.

The term “prevention” within the context of this invention refers not only to a complete prevention of the disease or one or more symptoms of the disease, but also to any partial or substantial prevention, attenuation, reduction, decrease or diminishing of the effect before or at early onset of disease.

The term treatment” within the context of this invention refers to any beneficial effect on progression of disease, including attenuation, reduction, decrease or diminishing of the pathological development after onset of disease.

The present invention has been described with reference to the specific embodiments but the content of the description comprises all modifications and substitutions, which can be brought by a person skilled in the art without extending beyond the meaning and purpose of the claims.

The invention will now be described by means of the following Examples, which should not be construed as in any way limiting the present invention. The Examples will refer to the Figures specified here below.

### EXAMPLES

### Example 1

### Production of Culling Fusion Proteins (CFPs)

Each of the culling fusion proteins contains an endocytosis domain, an exocytosis domain, and a culling domain (FIG. 2A). The DNA fragments coding for the Exocytosis Domain (ExDo), the Endocytosis Domain (EnDo), and the Culling Domain (CD, such as soluble receptors or monovalent antibodies that can bind to and neutralize therapeutic targets) can be generated and controlled in the appropriate expression vector by standard molecular biology technologies (PCR mutagenesis and amplification, DNA sequencing, restriction digestion). Expression vectors can be maintained in strain of E. coli during the cloning process but CFPs can be expressed in any kind of host cell (other bacteria, yeast, as well as insect, plant or mammalian cells).

In order to facilitate the generation of CFPs, a CFP-dedicated vector should contain a multiple cloning site at the 3′ and/or 5′ end of the sequence encoding the Exocytosis Domain (ExDo) and the Endocytosis Domain (EnDo), so that a Culling Domain (CD) can be easily cloned and expressed in-frame generating functional CFPs. These vectors, in order to direct CFPs through the secretion pathway, can also provide a heterologous secretion signal that results fused at N-terminus of the CFPs.

Once expressed, CFPs can be isolated from cell cultures using any technology known for protein purification (e.g. gel filtration, liquid/affinity chromatography).

Examples of protein sequences for CFPs directed against VEGF (SEQ ID NO: 11-14), TNFalpha (SEQ ID NO: 16-19), and IL-18 (SEQ ID NO: 21-24) are provided (FIG. 2B).

### Example 2

### In vitro Characterization of CFPs

Upon the construction, expression, and purification of the CFPs, their in vitro characterization involves preliminary studies for checking whether endocytosis, exocytosis, and target-binding domains retain their respective binding activities (i.e. for membrane-bound proteins triggering the endocytosis/exocytosis of the CFPs and the therapeutic target).

These studies can make use of recombinant or purified test proteins potentially interacting with CFPs to form complexes that can be detected with any appropriate method. At this scope, any technology, allowing a determination of protein-protein interactions that is reliable at least qualitatively, can be used with test proteins and the CFPs.

According to the chosen method, test proteins and CFPs may be used as such, complexed with membranes or antibodies, modified with a detectable label, and/or immobilized on a support. For example, CFPs can be prepared in a radioactive form, by iodinating CFPs with commercial kits (IODO-GEN; Pierce), or in a fluorescent form, by modifying CFPs with fluorescein isothiocyanate (FITC) according to manufacturer's instructions (Molecular Probes)

Protein microarrays, mass/NMR spectroscopy, affinity chromatography, fluorescence-based and antibody-based technologies (e.g. Western blot) are some examples of applicable methods. Such studies should also involve control proteins (e.g. Transferrin receptor, an un-/related ETT), the comparison between different conditions (e.g. binding activity at acid and neutral pH), allowing a quantitative evaluation of the binding parameters of the CFPs, such as the dissociation constant for different proteins.

Standard biochemical methods, such as immunoprecipitation or ELISA, can be used for confirming interactions between CFPs and ETT, or a cell component. For example, the extracellular region of the Transferrin receptor can be produced as described (Lawrence C M et al., 1999), and detection reagents such as monoclonal antibodies are commercially available (Research Diagnostics Inc).

CFPs directed against VEGF (SEQ ID NO: 11-14), TNFalpha (SEQ ID NO: 16-19), and IL-18 (SEQ ID NO: 21-24) can be tested and compared using detection reagents and kits commercially available (R&D Systems, Assay Designs Inc.).

### Example 3

### Cell-Based Assays

CFPs are designed and constructed to contain the minimal information allowing
the ETT binding,
the binding to the cell receptors, and
the recycling via receptor-mediated endocytosis and exocytosis.

In this context, the in vitro assay described in the previous paragraph are preliminary to cell binding assays for CFPs, which can be designed as equilibrium binding assay involving labeled CFPs added to cell cultures, so that immobilized CFPs can be measured. This assay, with appropriate modifications, can be carried out as described for differentiated hepatocytes or human colon carcinoma cells HT-29cl.19A (Sitaram M P and McAbee D, 1997).

The amount of CFPs immobilized on the cells can be measured, for example, with HT-29cl.19A cells grown filter discs can be mixed with various concentration of iodinated CFPs in presence of Ringer-HEPES buffer and of competing, non-labeled molecules (e.g. 0.2% serum Transferrin), or any other appropriate control molecule (e.g the ETT). The cells should be washed carefully and cell-associated radioactivity can be determined so that, by quantifying bound and unbound radioactivity and performing a Scatchard analysis, the specificity of the CFPs for cells can be determined from the saturation binding results.

Alternatively, a qualitative indication of the cell binding properties of CFPs can be obtained, for example, by incubating fluorescently- or radioactively-labeled CFPs with human CaCo cells grown in transparent inserts from a bicameral chamber (Costar) in the appropriate buffer (50 mM Na-MOPS, pH 7.4, 94 mM NaCl, 7.4 m M KCl, 0.74 mM MgCl2, 1.4 mM CaCl2). After 60 minutes at 37° C. with the labeled CFPs, cells can be washed with cold saline buffer and subsequently fixed in 3% glutaraldehyde. Internal and surface bound CFPs can be determined by measuring fluorescence in the cells by confocal microscopy, or by exposing the cells to a film. Labeled or unlabeled molecules, such as monoclonal antibodies against the ETT or the cell receptor, can be used as negative control.

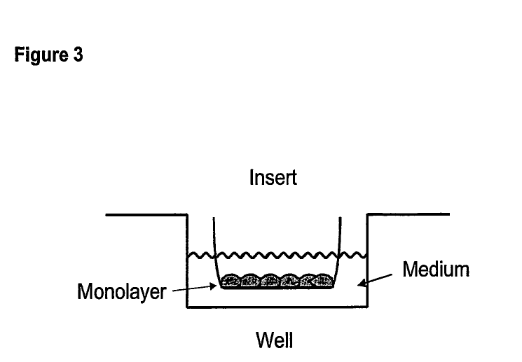

A further step towards the validation of CFPs is represented by assays demonstrating that CFPs are actively transported, via receptor-mediated endo- and exocytosis, through a monolayer of cells cultured in specific cell culture plates (FIG. 3).

Such assays, showing the trafficking of proteins through a monolayer and termed as transcytosis assays, involve the addition of non-/labeled CFPs (with or without the therapeutic target, or any other control molecule) to the cell culture medium in the “Insert” side. If CFPs are endocytosed and exocytosed after releasing the therapeutic target, at least a significant fraction of the added CFPs (but not a significant fraction of the therapeutic target) should be detected in the “Well” side by any appropriate analytical method.

Transcytosis assays involving pure or mixed cell cultures, which express Transferrin receptors and form monolayers with tight junction (preventing free passage of molecules through the monolayer), and labeled proteins are known in the literature for various cell types (Mikogami T et al., 1994; Fillebeen C et al., 1999; Megias L et al., 2000),

In an experimental design to test transcytosis of CFPs known in the literature (Shah D and Shen W C, 1996; Nunez M T et al., 1997), Caco-2 cells (ATCC number: HTB-37), that express Transferrin receptor and grow as a polarized membrane on microporous filters, are seeded in cell culture inserts containing porous flat bottom (e.g. Falcon Cell Culture Inserts) at a density not exceeding a 1/7^{th} of the surface area of the inserts, and cultured in regular 24 well tissue culture dishes. Caco-2 cells can be grown in Dulbecco's Minimal Essential Medium (DMEM) supplemented with 10% Fetal Bovine Serum (FBS). Once cell monolayers become confluent (after 10-15 days), tight junctions are correctly formed, but this feature can be tested by measuring a trans-epithelia electrical resistance (TEER) of at least 250 Ohm/cm² with a Volt-Ohm-meter.

After washing extensively cells with DMEM without FBS, the transcytosis experiment starts by adding the iodinated CFPs are to the buffer at the apical side in presence or absence of 100-fold excess of unlabelled CFPs or any other control molecule. At various time (0-6 hours), medium at the basolateral side are collected and equal volume of the collected samples are added back for replenishment. High amount of unlabeled transferrin can be added in the basolateral side to prevent reverse transcytosis of the trafficked CFPs. The radioactive proteins in the collected samples are subjected to TCA precipitation, and the radioactivity level in the precipitate can be measured with a Gamma counter. The intactness of the trafficked CFP can be analysed by SDS-PAGE and autoradiography. The specific transcytosis is the amount of the CFP transported through the monolayer after subtraction of the non-specific control, which is measured by counting trafficking in presence of 100-fold excess of unlabelled transferrin.

The effects of CFPs on the removal of a ETT can be also tested in a relevant animal model, wherein the ETT or a ETT-inducing compound is administered to the animal, or in a transgenic mice (e.g. the ETT is constituvely over-expressed). ELISA or other antibody-based assays performed on circulating liquids should allow determining the concentration of the CFP and/or of the ETT remaining in the circulation following the administration of CFPs or negative-control substances. Similar models are well known in the literature for several ETTs, and in particular the ones (VEGF, IL-18, TNFalpha) against which the CFPs disclosed in this application (SEQ ID NO: 11-14, 16-19, and 21-24) are directed for neutralizing their undesirable effects (e.g. promoting activity on the growth of endothelial cells for VEGF). The literature shows many different approaches for comparing the antagonistic, therapeutic, and pharmacokinetic activities amongst different CFPs or, between CFP and a known ETT antagonist (e.g. an anti-VEGF antibody compared to a VEGF-directed CFP). Further characterization of the biological and therapeutic activities of CFPs described in the present invention can be obtained by applying various in molecular biology technologies, such as two-dimensional gel electrophoresis or RNA interference.

TABLE I

| | | Proteins containing the | |
|---|---|---|---|
| Human ETT | | Culling Domain | |
| | SWISSPROT | | SWISSPROT |
| Name | Acc. No. | Name | Acc. No. |
| VEGF | P15692 | VEGFR-1 | P17498 |
| | | VEGFR-2 | P35968 |
| | | Neuropilin-1 | O14786 |
| EGF | P01133 | EGFR | P00533 |
| CCL5 (RANTES) | P13501 | CCR1 | P32246 |
| | | CCR5 | P32302 |
| CXCL12 (SDF-1) | P48601 | CXCR4 | P30991 |
| IFNgamma | P01579 | IFNgamma rec. | P15260 |
| TNFalpha | P01375 | TNF-R1 | P19438 |
| | | TNF-R2 | P20333 |
| IL-1alpha | P01583 | IL-1R | P14778 |
| | | IL-1 | P18510 |
| IL-4 | P05112 | IL-4R | P24394 |
| IL-18 | Q14116 | IL-18bp | O95998 |

TABLE II

| Amino | | More Preferred |
|---|---|---|
| Acid | Synonymous Group | Synonymous Groups |
| Ala | Gly, Thr, Pro, Ala, Ser | Gly, Ala |
| Arg | Asn, Lys, Gln, Arg, His | Arg, Lys, His |
| Asn | Glu, Asn, Asp, Gln | Asn, Gln |
| Asp | Glu, Asn, Asp, Gln | Asp, Glu |
| Cys | Ser, Thr, Cys | Cys |
| Gln | Glu, Asn, Asp, Gln | Asn, Gln |
| Glu | Glu, Asn, Asp, Gln | Asp, Glu |
| Gly | Ala, Thr, Pro, Ser, Gly | Gly, Ala |
| His | Asn, Lys, Gln, Arg, His | Arg, Lys, His |
| Ile | Phe, Ile, Val, Leu, Met | Ile, Val, Leu, Met |
| Leu | Phe, Ile, Val, Leu, Met | Ile, Val, Leu, Met |
| Lys | Asn, Lys, Gln, Arg, His | Arg, Lys, His |
| Met | Phe, Ile, Val, Leu, Met | Ile, Val, Leu, Met |
| Phe | Trp, Phe, Tyr | Tyr, Phe |
| Pro | Gly, Ala, Ser, Thr, Pro | Pro |
| Ser | Gly, Ala, Ser, Thr, Pro | Thr, Ser |
| Thr | Gly, Ala, Ser, Thr, Pro | Thr, Ser |
| Trp | Trp, Phe, Tyr | Trp |
| Tyr | Trp, Phe, Tyr | Phe, Tyr |
| Val | Met, Phe, Ile, Leu, Val | Met, Ile, Val, Leu |

### REFERENCES

Baker E N et al., Biochem Cell Biol, 80: 27-34, 2002.
Beisser P S et al., Curr Top Microbiol Immunol, 269: 203-34, 2002.
Bennett M J et al., Nature 403: 46-53, 2000.
Blobel C P, Inflamm Res, 51: 83-84, 2002.
Cleland et al., Crit Rev Ther Drug Carrier Syst, 10: 307-77, 1993.
Cleland J L et al., Curr Opin Biotechnol, 12: 212-219, 2001.
Cunningham, et al., Science, 244:1081-5, 1989.
Davies P S et al., Biochem J, 373:145-153, 2003.
Ehrlich Rand Lemonnier F A, Immunity, 13: 585-538, 2000.
Feder J N et al., Nature Genetics, 13: 399-408, 1996
Feder J N, Proc. Natl. Acad. Sci. USA, 95: 1472-1477, 1998.
Fillebeen C et al., J Biol Chem, 274: 7011-7017, 1999.
Gendel S M, Ann NY Acad SCI, 964: 87-98, 2002.
Gentz et al., Proc Natl Acad Sci USA, 86: 821-4, 1989.
Graddis T J et al., Curr Pharm Biotechnol, 3: 285-97, 2002.
Gustafsson C et al., Trends Biotechnol, 22: 346-53, 2004.
Kane J F, Curr Opin Biotechnol, 6: 494-500, 1995.
Jussila L and Alitalo K., Physiol Rev, 82: 673-700, 2002.
Lebron J A et al., Cell, 93: 111-123, 1998.
Lawrence C M et al., Science, 286: 779-782, 1999.
Lorenz H M and Kalden J R, Arthritis Res, 4 Suppl 3:S17-24, 2002.
Luo B and Prestwich G D, Exp Opin Ther Patents, 11: 1395-1410, 2001.
Makrides S C, Protein Expr Purif, 17: 183-202, 1999.
Marshall S A et al., Drug Disc Today, 8: 212-221, 2003
McAbee D D et al., Biochemistry 32: 13749-13760, 1993.
McCawley L J and Matrisian L M, Curr Opin Cell Biol, 13: 534-40, 2001.
Megias L et al., Int J Dev Biol, 44: 209-221, 2000.
Meilinger M et al., FEBS Lett, 360: 70-74, 1995.
Mikogami T et al., Am J Physiol, 267: G308-315, 1994
Murphy L R et al., Protein Eng, 13: 149-152, 2000.
Nakanishi K et al., Ann Rev Immun, 19: 423-474, 2001
Nunez M T et al., J Biol Chem, 272: 19425-19428, 1997.
Park E et al., J Drug Targ, 6: 53-64, 1998.
Pillai O and Panchagnula R, Cur Opin Chem Biol, 5: 447-451, 2001.
Pillay C S et al., Biochem J, 363: 417-429, 2002.
Pini A and Bracci L, Curr Protein Pept Sci, 1: 155-169, 2000
Qian Z M et al., Pharmacol Rev, 54: 561-587, 2002.
Richardson D R and Ponka P, Biochim Biophys Acta, 1331: 1-40, 1997.
Rogov S I and Nekrasov A N, Protein Eng, 14: 459-463, 2001.
Sachse M et al., Histochem Cell Biol, 117: 91-104, 2002.
Schellekens H, Nat Rev Drug Disc, 1: 457-462, 2002.
Shah D., and Shen W C, J Pharm Sci, 85: 1306-1311, 1996.
Sitaram M P and McAbee D, Biochem.J, 323: 815-822, 1997
Swaan P W, Pharm Res, 15: 826-34, 1998.
Terpe K, Appl Microbiol Biotechnol, 60: 523-33, 2003.
Vasserot A P et al., Drug Disc Today, 8: 118-126, 2003.
Wilson et al., Cell, 37: 767-78, 1994.
Wong H and Schryvers A B, Adv Exp Med Biol, 443:101-106, 1998.

1-22. (canceled)

23. A chimeric protein comprising:
a) a recycling domain capable of binding a human cell surface receptor and formed by an Exocytosis Domain and an Endocytosis Domain; and
b) a protein domain binding an Extracellular Therapeutic Target.

24. The chimeric protein of claim 23, wherein the human cell surface receptor is human Transferrin receptor and the Endocytosis Domain is the alpha1-alpha2 domain of human HFE protein or human deltaN-Lactoferrin.

25. The chimeric protein of claim 24, wherein the Exocytosis Domain is the alpha3 domain of human HFE protein.

26. The chimeric protein of claim 25, wherein the amino acid sequence comprises SEQ ID NO: 4, SEQ ID NO: 5, SEQ ID NO: 6, or SEQ ID NO: 7.

27. The chimeric protein of claim 23, wherein the protein domain binds an Extracellular Therapeutic Target selected from: a cytokine, a chemokine, a hormone, a growth factor, an immunoglobulin, a glycolipid, a glycosaminoglycan, a nucleic acid, a viral protein, a bacterial protein, or a synthetic organic molecule.

28. The chimeric protein of claim 23, wherein the protein domain binding the Extracellular Therapeutic Target is selected from: an extracellular region of a membrane-bound protein, a secreted protein, a viral protein, an antigen binding domain of an antibody, or one or more selected domain of such protein sequences.

29. The chimeric protein of claim 23, further comprising an amino acid sequence of a heterologous protein, said amino acid sequence of a heterologous protein being other than the sequences of the proteins containing the Exocytosis Domain, the Endocytosis Domain, and the protein domain binding an Extracellular Therapeutic Target.

30. The chimeric protein of claim 29 further comprising a heterologous signal peptide.

31. The chimeric protein of claim 30, wherein said chimeric protein comprises a protein domain binding VEGF as Extracellular Therapeutic Target and the sequence corresponding to any of SEQ ID NOs: 11, 12, 13 or 14.

32. The chimeric protein of claim 30, wherein said chimeric protein comprises a protein domain binding TNFalpha as Extracellular Therapeutic Target and the sequence corresponding to any of SEQ ID NOs: 16, 17, 18 or 19.

33. The chimeric protein of claim 30, wherein said chimeric protein comprises a protein domain binding IL-18 as Extracellular Therapeutic Target and the sequence corresponding to any of SEQ ID NOs: 21, 22, 23 or 24.

34. The chimeric protein of claim 23, wherein the Exocytosis Domain, the Endocytosis Domain, and the protein domain binding an Extracellular Therapeutic Target are active mutants of the corresponding natural sequence.

35. The chimeric protein of claim 23, wherein said protein is in the form of an active fraction, precursors, salt, derivative, conjugate, or complex.

36. A composition of matter comprising:
a) an isolated DNA molecule encoding a chimeric protein comprising a recycling domain capable of binding a human cell surface receptor and formed by an Exocytosis Domain and an Endocytosis Domain; and a protein domain binding an Extracellular Therapeutic Target;
b) expression vectors comprising a DNA molecule encoding a chimeric protein comprising a recycling domain capable of binding a human cell surface receptor and formed by an Exocytosis Domain and an Endocytosis Domain; and a protein domain binding an Extracellular Therapeutic Target, wherein expression of said DNA is under the control of a promoter;
c) a host cell transformed with expression vectors comprising a DNA molecule encoding a chimeric protein comprising a recycling domain capable of binding a human cell surface receptor and formed by an Exocytosis Domain and an Endocytosis Domain; and a protein domain binding an Extracellular Therapeutic Target, wherein expression of said DNA is under the control of a promoter;
d) purified preparations of a chimeric protein comprising a recycling domain capable of binding a human cell surface receptor and formed by an Exocytosis Domain and an Endocytosis Domain; and a protein domain binding an Extracellular Therapeutic Target;
e) a pharmaceutical composition comprising a recycling domain capable of binding a human cell surface receptor and formed by an Exocytosis Domain and an Endocytosis Domain; and a protein domain binding an Extracellular Therapeutic Target; or
f) a pharmaceutical composition comprising a host cell transformed with expression vectors comprising a DNA molecule encoding a chimeric protein comprising a recycling domain capable of binding a human cell surface receptor and formed by an Exocytosis Domain and an Endocytosis Domain; and a protein domain binding an Extracellular Therapeutic Target, wherein expression of said DNA is under the control of a promoter.

37. A method for the treatment or prevention of a disease, comprising the administration of an effective amount of a chimeric protein comprising a recycling domain capable of binding a human cell surface receptor and formed by an Exocytosis Domain and an Endocytosis Domain; and a protein domain binding an Extracellular Therapeutic Target or a host cell transformed with expression vectors comprising a DNA molecule encoding a chimeric protein comprising a recycling domain capable of binding a human cell surface receptor and formed by an Exocytosis Domain and an Endocytosis Domain; and a protein domain binding an Extracellular Therapeutic Target to an individual.
